(19) United States
(12) Patent Application Publication
(10) Pub. No.: US 2023/0323468 A1
(43) Pub. Date: Oct. 12, 2023

(54) IMMUNE GENE SIGNATURES IN MUSCLE INVASIVE BLADDER CANCER

(71) Applicant: H. Lee Moffitt Cancer Center and Research Institute, Inc., Tampa, FL (US)

(72) Inventors: Roger Li, Tampa, FL (US); James J. Mulé, Odessa, FL (US); Anders Erik Berglund, Tampa, FL (US)

(21) Appl. No.: 18/023,261

(22) PCT Filed: Aug. 27, 2021
(86) PCT No.: PCT/US2021/048028

§ 371 Date: Feb. 24, 2023

Related U.S. Application Data
(60) Provisional application No. 63/071,320, filed on Aug. 27, 2020.

(51) Int. Cl. C12Q 1/6886

(52) U.S. Cl. CPC C12Q 1/6886; C12Q 2600/158

(57) ABSTRACT

This invention relates to methods for selecting a treatment and optionally treating subjects with muscle-invasive bladder cancer based on tumor expression levels of chemokines, cytotoxic genes, and/or dendritic cell genes.

### CLAIM OF PRIORITY

This application claims the benefit of U.S. Patent Application Ser. No. 63/071,320, filed on Aug. 27, 2020. The entire contents of the foregoing are hereby incorporated by reference.

### TECHNICAL FIELD

This invention relates to methods of selecting treatment for, and optionally treating, subjects with Muscle Invasive Bladder Cancer (MIBC).

### BACKGROUND

In 2020, there were 81,400 new cases of bladder cancer, and there are estimated to be approximately 18,000 deaths. Bladder cancer is the fourth most common cancer in men, and mainly occurs in those over the age of 55. Urothelial carcinoma is the most common type of bladder cancer in the United States. Chemotherapy and surgery are the traditional treatment options, and both pembrolizumab (anti-PD-1) and atezolizumab (anti-PD-L1) are FDA approved for the treatment of patients with urothelial carcinoma that has spread beyond the bladder. Despite having revolutionized the treatment paradigm for muscle invasive and metastatic bladder cancers, response rates to immune checkpoint blockade (ICB) remain insufficient.^{1,2} Atezolizumab can cause severe and fatal immune-mediated adverse reactions that may involve any organ system including immune-mediated pneumonitis, hepatitis, colitis, or endocrinopathies. Because of their severity, immune-mediated adverse reactions can limit therapy and require immunosuppressive treatment.

### SUMMARY

The present invention is based, at least in part, on the discovery of gene signatures that predict the likelihood of relapse. Expression levels of these genes can be used to optimize or select treatment and predict survival in subjects with muscle invasive bladder cancer (MIBC). As shown herein, a high score of 12-Chemokine GES is correlated with improved overall survival in stage IV patients with MIBC.

Provided herein are methods for treating a subject who has muscle-invasive bladder cancer (MIBC). The methods include obtaining cells from the tumor; determining expression levels of CCL2, CCL3, CCL4, CCL5, CCL8, CCL18, CCL19, CCL21, CXCL9, CXCL10, CXCL11, and CXCL13 in the tumor cells; comparing the tumor gene expression levels to reference gene expression levels; selecting for the subject a first line treatment comprising surgical resection and immunotherapy and/or adjuvant chemotherapy if tumor gene expression levels are above the reference gene expression levels.

In some embodiments, determining gene expression levels comprises determining protein levels.

In some embodiments, determining gene expression levels comprises determining mRNA levels.

In some embodiments, the immunotherapy comprises administering to the subject dendritic cells or peptides with adjuvant, a DNA-based vaccine, cytokines, cyclophosphamide, anti-interleukin-2R immunotoxin, or a checkpoint inhibitor.

In some embodiments, the checkpoint inhibitor is an inhibitor of PD-1 signaling, preferably an antibody that binds to PD-1, CD40, or PD-L1, or an inhibitor of Tim3 or Lag3, preferably an antibody that binds to Tim3 or Lag3, or an antibody that binds to CTLA-4.

In some embodiments, the adjuvant chemotherapy comprises administering to the subject a platinum-based chemotherapeutic, e.g., cisplatin, carboplatin or oxaliplatin; paclitaxel, and/or gemcitabine; cisplatin/carboplatin (GC); Pembrolizumab; or methotrexate, vinblastine, doxorubicin, and cisplatin (MVAC).

In some embodiments, comparing expression levels comprises calculating an expression score that is a weighted average of the mRNA expression levels of CCL2, CCL3, CCL4, CCL5, CCL8, CCL18, CCL19, CCL21, CXCL9, CXCL10, CXCL11, and CXCL13, and comparing the expression score to a reference expression score.

In some embodiments of the methods described herein, the subject is a human.

In some embodiments of the methods described herein, the cancerous tumor of the bladder is, or is not, urothelial carcinoma (also known as transitional cell carcinoma (TCC)), squamous cell carcinoma, or adenocarcinoma.

In some embodiments comparing gene expression levels comprises calculating an expression score based on the gene expression levels, e.g., based on the weighted average of the gene expression levels, and comparing the expression score to a reference expression score.

A “subject” as described herein can be any subject having a proliferative disorder. For example, the subject can be any mammal, such as a human, including a human cancer patient. Exemplary nonhuman mammals include a nonhuman primate (such as a monkey or ape), mouse, rat, goat, cattle, pig, horse, sheep, cat, and dog.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H. High 12-chemokine score (12-CK) is associated with the presence of tertiary lymphoid structures (TLS) in the tumor microenvironment (TME) and a more robust peritumoral inflammatory response. (A) H&E and immunohistochemistry (IHC) stains were performed against CD4, CD8, CD20, and LAMP3 to define populations of CD4+ T lymphocytes, CD8+ T lymphocytes, CD20+ B lymphocytes, and activated dendritic cells in the TME, respectively. Type III tertiary lymphoid structures, consisting of prominent B-cell follicles with germinal center-like structures and discrete T-cell zones, were found within tumors as well as at the tumor invasive front. (B) Higher densities of CD4+ T lymphocytes (p=0.1), CD8+ T lymphocytes (p=0.02), B lymphocytes (p=0.008), and activated dendritic cells (p=0.047) were found in the 12CK-High TME. (C) High 12-CK scores were furthermore related to elevated immune scores, but not stromal scores as delineated by the gene signature-based deconvolution method xCell. (D-E) Findings using the deconvolution tool xCell corresponded with IHC findings, demonstrating higher expression levels of CD4+ T lymphocytes, CD8+ T lymphocytes, B lymphocytes, and activated dendritic cell transcriptomic signatures (D, top), and further exploration using xCell demonstrated heightened signatures related to immune cells found within the adaptive (D, bottom) and (E) innate immune responses. (F) Gene set enrichment analysis demonstrating elevated expression of pathways in both the innate and adaptive immune response found within the 12CK-High TME. (G) The 12-CK scores were found to be independent of traditional prognostic indicators, such as pathologic (left) T-staging and (right) N-staging. (H) The 12-CK scores were also found to be independent of the receipt of neoadjuvant chemotherapy . . . 12-CK—12-Chemokine score; TLS—Tertiary lymphoid structure; GC—germinal center; * p<0.05; ** p<10 0.01; *** p<0.001; **** p<0.0001.

FIGS. 2A-H. The prognostic and predictive implications of 12-CK score. Kaplan-Meier survival analyses revealed improved overall survival (OS, HR 0.55, p=0.03) (A), disease-specific survival (DSS, HR 0.25, p=0.003) (B), and progression-free survival (PFS, HR 0.25, p=0.003) (C) in 12CK-High muscle invasive bladder cancer patients treated with radical cystectomy at Moffitt Cancer Center. The favorable prognosis in OS (HR 0.59, p=0.010) (D), DSS (HR 0.40, p=0.002) (E), and PFS (HR 0.55, p=0.007) (F) were confirmed in patients from TCGA. (G) From the IMVIGOR-210 study testing the efficacy of atezolizumab in chemotherapy refractory locally advanced or metastatic bladder cancer patients, complete responders were found to have significantly higher 12CK scores than the other cohorts. (H) Stratified by the 12-CK score, 12CK-High patients were found to have an 11.2 mo overall survival benefit after treatment using atezolizumab.

### DETAILED DESCRIPTION

To date, the search for predictive biomarkers and strategies to augment clinical response have largely focused on the T cell compartment.³ Emerging evidence from immune correlative studies in sarcoma and melanoma ICB trials demonstrated enhanced efficacy in tumors harbouring tertiary lymphoid structures (TLS)⁴⁻⁶—lymph node like aggregates in the tumor microenvironment (TME) postulated to be 1) the gateway of naïve lymphocyte infiltration and 2) privileged sites for coordinated tumor antigen presentation and lymphocyte priming, differentiation, and proliferation.⁵⁻⁶ Further in-depth analyses of these TLS-containing tumors revealed strong infiltration of B lineage cells, T cells, monocytic cells (including myeloid dendritic cells) and endothelial cells.⁷ Previously, a 12-chemokine metagene (12CK) transcriptomic signature (CCL2, CCL3, CCL4, CCL5, CCL8, CCL18, CCL19, CCL21, CXCL9, CXCL10, CXCL11, and CXCL13) was described that reflects strong intratumoral immune chemotactic signalling and accurately predicts the presence of TLSs in colorectal carcinoma and melanoma.^{8,9}

The present study correlated high 12CK signature (12CK-High) with formation of TLS in the context of muscle invasive bladder cancer (MIBC). We then sought to define the prognostic implications of 12CK-High in MIBC and its ability to predict response to ICB.

As shown herein, profiling gene signatures predicts the presence of infiltrating immune cells, and expression levels of these genes can be used to assign a prognosis and select or optimize treatment in subjects with MICB.

Three important findings emerged from the current study: 12CK-High scores corresponded with formation of TLS in the TME; favourable prognosis in surgically treated MIBC patients; and CR in atezolizumab-treated patients. That the 12CK-High score corresponded with TLS formation in the context of MIBC recapitulated similar findings in other cancer types^{8,9}, and firmly establishes it as a useful tool reflecting effective anti-tumor immunity.⁷ Additionally, heightened peritumoral immune response as manifested by increased tumor-infiltrating CD8+ T cells has previously been associated with favourable prognosis in MIBC patients.¹¹ The search for biomarkers predicting response to ICB has evolved from the examination of tumor intrinsic factors such as mutational burden and molecular subtypes², to T cell infiltration and phenotypes³, to a more comprehensive appreciation of the different components of the TME.^{4-6,12-14} The findings herein show the 12CK gene signature to be a clinically actable biomarker for predicting response to ICB. With ICB's expanding indication in the treatment of bladder cancer diagnosed at various clinical stages^{1,2,15}, the 12CK signature can be used as an important tool to refine patient selection for ICB and adjuvant chemotherapy treatment.

Presently, subjects with MIBC are typically treated with surgical resection, and adjuvant chemotherapy and immunotherapy are reserved for second line treatment after a subject has relapsed. The present methods can be used to identify subjects who have a low likelihood of long-term survival, e.g., who are likely to relapse, and selecting those subjects for treatment with adjuvant chemotherapy and/or immunotherapy as an initial treatment, e.g., at the time of initial resection, e.g., before, during, or within 1, 2, 4, 6, 8, 12 or 24 hours of resection hours.

### Methods of Assigning a Prognosis or Predicting Survival

The methods can be used to select a treatment, e.g., to select a treatment regime including adjuvant chemotherapy or immunotherapy after surgery for a subject. In addition, the methods described herein can be used for, e.g., to assist in, assigning a prognosis or predicting survival in a subject who has MICB.

Methods for diagnosing a subject with MICB are known in the art.

### Assays, References, and Samples

The methods described herein include determining levels of selected chemokine genes. In some embodiments, all of the genes listed in the tables below are evaluated. In some embodiments, two, three, four, five, six, seven, eight, nine, ten, eleven, or all twelve of the listed genes are evaluated. Although the terminology “genes” is used herein, in some embodiments, the methods include detecting levels of the proteins encoded by the listed genes. In some embodiments, the methods include detecting transcript (mRNA) levels.

### Chemokines

Chemokines are secreted proteins involved in immunoregulatory and inflammatory processes. The chemokines used in the present methods are as follows:

Chemokines

| | | GenBank Acc. | GenBank Acc. |
|---|---|---|---|
| Gene | | No .: | No .: |
| Symbol | Gene Name | Nucleic Acid | Protein |
| CCL2 | chemokine (C-C motif) ligand 2 | NM_002982.3 | NP_002973.1 |
| CCL3 | chemokine (C-C motif) ligand 3 | NM_002983.2 | NP_002974.1 |
| CCL4 | chemokine (C-C motif) ligand 4 | NM_002984.2 | NP_002975.1 |
| CCL5 | chemokine (C-C motif) ligand 5 | NM_002985.2 | NP_002976.2 |
| CCL8 | chemokine (C-C motif) ligand 8 | NM_005623.2 | NP_005614.2 |
| CCL18 | chemokine (C-C motif) ligand 18 | NM_002988.2 | NP_002979.1 |
| | (pulmonary and activation-regulated) | | |
| CCL19 | chemokine (C-C motif) ligand 19 | NM_006274.2 | NP_006265.1 |
| CCL21 | chemokine (C-C motif) ligand 21 | NM_002989.2 | NP_002980.1 |
| CXCL9 | chemokine (C-X-C motif) ligand 9 | NM_002416.1 | NP_002407.1 |
| CXCL10 | chemokine (C-X-C motif) ligand 10 | NM_001565.2 | NP_001556.2 |
| CXCL11 | chemokine (C-X-C motif) ligand 11 | NM_005409.4 | NP_005400.1 |
| CXCL13 | chemokine (C-X-C motif) ligand 13 | NM_006419.2 | NP_006410.1 |

In some embodiments, the methods include assaying the presence or levels of chemokine mRNA or proteins in the sample. The presence and/or level of a protein can be evaluated using methods known in the art, e.g., using quantitative immunoassay methods. The presence and/or level of an mRNA can be evaluated using methods known in the art, e.g., Northern blotting or quantitative PCR methods, e.g., RT-PCR. In some embodiments, high throughput methods, e.g., protein or gene chips as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of chemokine proteins as described herein.

In some embodiments, the methods include assaying levels of one or more control genes or proteins, and comparing the level of expression of the chemokine genes or proteins to the level of the control genes or proteins, to normalize the levels of the chemokine genes or proteins. Suitable endogenous control genes includes a gene whose expression level should not differ between samples, such as a housekeeping or maintenance gene, e.g., 18S ribosomal RNA; beta Actin; Glyceraldehyde-3-phosphate dehydrogenase; Phosphoglycerate kinase 1; Peptidylprolyl isomerase A (cyclophilin A); Ribosomal protein L13a; large Ribosomal protein P0; Beta-2-microglobulin; Tyrosine 3-monooxygenase/tryptophan 5-monooxygenase activation protein, zeta polypeptide; Succinate dehydrogenase; Transferrin receptor (p90, CD71); Aminolevulinate, delta-, synthase 1; Glucuronidase, beta; Hydroxymethyl-bilane synthase; Hypoxanthine phosphoribosyltransferase 1; TATA box binding protein; and/or Tubulin, beta polypeptide.

Generally speaking, the methods described herein can be performed on cells from a tumor. The cells can be obtained by known methods, e.g., during a biopsy (such as a core needle biopsy or cystoscopic biopsy), or during a surgical procedure to remove all or part of the tumor (e.g., transurethral resection of the bladder tumor (TURBT), or partial or radical (complete) cystectomy). The cells can be used fresh, frozen, fixed, and/or preserved, so long as the mRNA or protein that is to be assayed is maintained in a sufficiently intact state to allow accurate analysis.

In some embodiments of the methods described herein, the levels of the chemokine genes in the tumor sample can be compared individually to levels in a reference. The reference levels can represent levels in a subject who has a good prognosis, or a long predicted survival time (e.g., 2 years or more). Alternatively, reference levels can represent levels in a subject who has a poor prognosis, or a shorter predicted survival time (e.g., less than 2 years). In some embodiments, the reference levels represent a threshold, and a level in the tumor that is above the threshold reference level indicates that the subject has a good prognosis, or a long predicted survival time (e.g., 2 years or more), and levels below the threshold reference level indicates that the subject has a poor prognosis, or a shorter predicted survival time (e.g., less than 2 years).

In some embodiments, the reference levels can represent levels in a subject who has lymphoid like structures present in the tumor, or is predicted to respond to immunotherapy. Alternatively, reference levels can represent levels in a subject who lacks tumor lymphoid structures, or is predicted to have no or a poor response to immunotherapy. In some embodiments, the reference levels represent a threshold, and a level in the tumor that is above the threshold reference level indicates that the subject has tumor lymphoid structures, or is predicted to respond to immunotherapy, and levels below the threshold reference level indicates that the subject lacks lymphoid structures and is predicted to have no or poor response to immunotherapy. In subjects who are predicted to have tumor lymphoid structures, or who are predicted to respond to immunotherapy, the methods can further include administering an immunotherapy for those subjects, or selecting or recommending a treatment including an immunotherapy for those subjects.

In some embodiments of the methods described herein, values representing the levels of the chemokine genes can be summed to produce a “tumor chemokine gene score” that can be compared to a reference chemokine gene score, wherein a tumor chemokine gene score that is above the reference chemokine gene score indicates that the subject has a long predicted survival time (e.g., 2 years or more) or is predicted to have a positive response to immunotherapy, and a chemokine gene score below the reference score indicates that the subject has a shorter predicted survival time (e.g., less than 2 years), or is predicted to have no or a poor response to immunotherapy.

For example, in some embodiments, the expression levels of each of the evaluated genes can be assigned a value (e.g., a value that represents the expression level of the gene, e.g., normalized to an endogenous control gene as described herein). That value (optionally weighted to increase or decrease its effect on the final score) can be summed to produce a chemokine gene score. One of skill in the art could optimize such a method to determine an optimal algorithm for determining a chemokine gene score.

The methods described herein can include determining levels (or scores) for all of the 12 chemokine genes. In some embodiments all of the genes in each set are evaluated, but in some embodiments a subset of one or all of the sets is evaluated.

One of skill in the art will appreciate that references can be determined using known epidemiological and statistical methods, e.g., by determining a chemokine gene score, or chemokine gene protein or mRNA levels, in tumors from an appropriate cohort of subjects, e.g., subjects with the same type of cancer as the test subject and a known prognosis (e.g., good or poor) or predicted survival time (e.g., less than 2 years, or 2 years or more).

### Immunotherapy

In some embodiments, the methods include selecting and optionally administering a first line treatment comprising an immunotherapy, e.g., comprising administering to the subject one or more therapies that promote anti-cancer immunity, including administering one or more of: dendritic cells or peptides with adjuvant, DNA-based vaccines, cytokines (e.g., IL-2), cyclophosphamide, anti-interleukin-2R immunotoxins, and/or antibodies (e.g., immune checkpoint blockage agents).

In some embodiments, the methods can include administering a checkpoint inhibitor, e.g., an inhibitor of PD-1 signaling, e.g., an antibody that binds to PD-1, CD40, or PD-L1, or an inhibitor of Tim3 or Lag3, e.g., an antibody that binds to Tim3 or Lag3, or an antibody that binds to CTLA-4.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab (see Topalian, et al., NEJM. 366(26): 2443-2454 (2012) and WO2013/173223A1), avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; and U.S. Pat. No. 8,591,900, including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary CTLA-4 antibodies that can be used in the methods described herein include those that bind to human CTLA-4; exemplary CTLA-4 protein sequences are provided at NCBI Acc No. NP_005205.2. Exemplary antibodies include those described in Tarhini and Iqbal, Onco Targets Ther. 3:15-25 (2010); Storz, MAbs. 2016 Jan; 8(1): 10-26; US2009025274; U.S. Pat. Nos. 7,605,238; 6,984,720; EP1212422; U.S. Pat. Nos. 5,811,097; 5,855,887; 6,051,227; 6,682,736; EP1141028; and U.S. Pat. No. 7,741,345; and include ipilimumab (see Tarhini and Iqbal, Onco Targets Ther. 3:15-25 (2010) and U.S. Pat. No. 7,741,345), Tremelimumab, and EPR1476.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A, see Powles et al., Nature. 2014 Nov 27;515(7528):558-62), Avelumab (Bavencio), and Durvalumab (Imfinzi, MEDI-4736).

Exemplary anti-Tim3 (also known as hepatitis A virus cellular receptor 2 or HAVCR2) antibodies that can be used in the methods described herein include those that bind to human Tim3; exemplary Tim3 sequences are provided at NCBI Accession No. NP_116171.3. Exemplary antibodies are described in WO2016071448; U.S. Pat. No. 8,552,156; and US Pub. Nos. 20180298097; 20180251549; 20180230431; 20180072804; 20180016336; 20170313783; 20170114135; 20160257758; 20160257749; 20150086574; and 20130022623, and include LY3321367, DCB-8, MBG453 and TSR-022.

Exemplary anti-Lag3 antibodies that can be used in the methods described herein include those that bind to human Lag3; exemplary Lag3 sequences are provided at NCBI Accession No. NP_002277.4. Exemplary antibodies are described in Andrews et al., Immunol Rev. 2017 Mar; 276(1):80-96; Antoni et al., Am Soc Clin Oncol Educ Book. 2016; 35:e450-8; US Pub. Nos. 20180326054; 20180251767; 20180230431; 20170334995; 20170290914; 20170101472; 20170022273; 20160303124, and include BMS-986016.

The methods can also include administering one or more anti-CD137 antibodies, e.g., urelumab (BMS-663513) and PF-05082566 (see Yonezawa et al., Clin Cancer Res. 2015 Jul 15; 21(14): 3113-3120).

See, e.g., Kruger et al., “Immune based therapies in cancer,” Histol Histopathol. 2007 Jun;22(6):687-96; Eggermont et al., “Anti-CTLA-4 antibody adjuvant therapy in melanoma,” Semin Oncol. 2010 Oct;37(5):455-9; Klinke D J 2nd, “A multiscale systems perspective on cancer, immunotherapy, and Interleukin-12,” Mol Cancer. 2010 Sep 15;9:242; Alexandrescu et al., “Immunotherapy for melanoma: current status and perspectives,” J Immunother. 2010 Jul-Aug;33(6):570-90; Moschella et al., “Combination strategies for enhancing the efficacy of immunotherapy in cancer patients,” Ann N Y Acad Sci. 2010 Apr;1194:169-78; Ganesan and Bakhshi, “Systemic therapy for melanoma,” Natl Med J India. 2010 Jan-Feb;23(1):21-7; Golovina and Vonderheide, “Regulatory T cells: overcoming suppression of T-cell immunity,” Cancer J. 2010 Jul-Aug;16(4):342-7. In some embodiments, the methods include administering a composition comprising tumor-pulsed dendritic cells, e.g., as described in WO2009/114547 and references cited therein. See also Shiao et al., Genes & Dev. 2011. 25: 2559-2572.

### Pseudoprogression

Pseudoprogression is a common phenomenon when treating with immune checkpoint blockade where the tumor appears to progress in size radiologically prior to shrinking. In a phase II study of Atezolizumab in urothelial carcinoma, 20 of 121 patients (17%) experienced a target lesion reduction after tumor progression. This phenomenon is also seen in melanoma and non small-cell lung cancer, as well as head and neck cancers, at rates of up to 10%. The present methods can be used to determine which patients with progressive disease likely have pseudoprogression and should continue to receive checkpoint blockade therapies even after apparent progression.

### Adjuvant Chemotherapy

In some embodiments, the methods include selecting and optionally administering a first line treatment comprising an adjuvant chemotherapy, e.g., comprising administering to the subject one or more of a platinum-based chemotherapeutic, e.g., cisplatin, carboplatin or oxaliplatin; paclitaxel, and/or gemcitabine; cisplatin/carboplatin (GC); Pembrolizumab; or methotrexate, vinblastine, doxorubicin, and cisplatin (MVAC). See, e.g., Advanced Bladder Cancer Overview Collaboration, Cochrane Database Syst Rev. 2005 Apr 18;(2):CD005246; Del Bene et al., Front Oncol. 2018; 8: 463; Yin et al., Oncologist. 2016 Jun;21(6):708-15;

### Computer Software/Hardware

Standard computing devices and systems can be used and implemented to perform the methods described herein. Computing devices include various forms of digital computers, such as laptops, desktops, mobile devices, workstations, personal digital assistants, servers, blade servers, mainframes, and other appropriate computers. In some embodiments, the computing device is a mobile device, such as personal digital assistant, cellular telephone, smartphone, tablet, or other similar computing device. The components described herein, their connections and relationships, and their functions, are meant to be exemplary only, and are not meant to limit implementations of the inventions described and/or claimed in this document.

Computing devices typically include one or more of a processor, memory, a storage device, a high-speed interface connecting to memory and high-speed expansion ports, and a low speed interface connecting to low speed bus and storage device. Each of the components are interconnected using various busses, and can be mounted on a common motherboard or in other manners as appropriate. The processor can process instructions for execution within the computing device, including instructions stored in the memory or on the storage device to display graphical information for a GUI on an external input/output device, such as a display coupled to a high speed interface. In other implementations, multiple processors and/or multiple buses can be used, as appropriate, along with multiple memories and types of memory. Also, multiple computing devices can be connected, with each device providing portions of the operations (e.g., as a server bank, a group of blade servers, or a multi-processor system).

The memory stores information within the computing device. In some embodiments, the memory is a computer-readable medium. In one implementation, the memory is a volatile memory unit or units. In another implementation, the memory is a non-volatile memory unit or units.

The storage device is capable of providing mass storage for the computing device. In one implementation, the storage device is a computer-readable medium. In various different implementations, the storage device can be a floppy disk device, a hard disk device, an optical disk device, or a tape device, a flash memory or other similar solid state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory, the storage device, memory on processor, or a propagated signal.

The high speed controller manages bandwidth-intensive operations for the computing device, while the low speed controller manages lower bandwidth-intensive operations. Such allocation of duties is exemplary only. In one implementation, the high-speed controller is coupled to memory, the display (e.g., through a graphics processor or accelerator), and to high-speed expansion ports, which can accept various expansion cards (not shown). In the implementation, the low-speed controller is coupled to a storage device and low-speed expansion port. The low-speed expansion port, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device can be implemented in a number of different forms. For example, it can be implemented as a standard server, or multiple times in a group of such servers. It can also be implemented as part of a rack server system. In addition, it can be implemented in a personal computer such as a laptop computer. Alternatively, components from the computing device can be combined with other components in a mobile device. Each of such devices can contain one or more computing devices, and an entire system can be made up of multiple computing devices communicating with each other.

The computing device typically includes a processor, memory, an input/output device such as a display, a communication interface, and a transceiver, among other components. The device can also be provided with a storage device, such as a microdrive or other device, to provide additional storage. Each of these components are interconnected using various buses, and several of the components can be mounted on a common motherboard or in other manners as appropriate.

The processor can process instructions for execution within the computing device, including instructions stored in the memory. The processor can also include separate analog and digital processors. The processor can provide, for example, for coordination of the other components of the device, such as control of user interfaces, applications run by the device, and wireless communication by the device.

The processor can communicate with a user through control interface and display interface coupled to a display. The display can be, for example, a TFT LCD display or an OLED display, or other appropriate display technology. The display interface can comprise appropriate circuitry for driving the display to present graphical and other information to a user. The control interface can receive commands from a user and convert them for submission to the processor. In addition, an external interface can be provide in communication with the processor, so as to enable near area communication of device with other devices. External interface can provide, for example, for wired communication (e.g., via a docking procedure) or for wireless communication (e.g., via Bluetooth or other such technologies).

The memory stores information within the computing device. In one implementation, the memory is a computer-readable medium. In one implementation, the memory is a volatile memory unit or units. In another implementation, the memory is a non-volatile memory unit or units. Expansion memory can also be provided and connected to the device through an expansion interface, which can include, for example, a SIMM card interface. Such expansion memory can provide extra storage space for device, or can also store applications or other information for the device. Specifically, expansion memory can include instructions to carry out or supplement the processes described above, and can include secure information also. Thus, for example, expansion memory can be provided as a security module for the device, and can be programmed with instructions that permit secure use of the device. In addition, secure applications can be provided via the SIMM cards, along with additional information, such as placing identifying information on the SIMM card in a non-hackable manner.

The memory can include for example, flash memory and/or MRAM memory, as discussed below. In one implementation, a computer program product is tangibly embodied in an information carrier. The computer program product contains instructions that, when executed, perform one or more methods, such as those described above. The information carrier is a computer- or machine-readable medium, such as memory, expansion memory, memory on processor, or a propagated signal.

The device can communicate wirelessly through a communication interface, which can include digital signal processing circuitry where necessary. The communication interface can provide for communications under various modes or protocols, such as GSM voice calls, SMS, EMS, or MMS messaging, CDMA, TDMA, PDC, WCDMA, CDMA2000, or GPRS, among others. Such communication can occur, for example, through a radio-frequency transceiver. In addition, short-range communication can occur, such as using a Bluetooth, WiFi, or other such transceiver.

The device can also communication audibly using audio codec, which can receive spoken information from a user and convert it to usable digital information. Audio codex can likewise generate audible sound for a user, such as through a speaker, e.g., in a handset of device. Such sound can include sound from voice telephone calls, can include recorded sound (e.g., voice messages, music files, etc.) and can also include sound generated by applications operating on device.

The computing device can be implemented in a number of different forms, as shown in the figure. For example, it can be implemented as a cellular telephone. It can also be implemented as part of a smartphone, tablet, personal digital assistant, or other similar mobile device.

Where appropriate, the systems and the functional operations described in this specification can be implemented in digital electronic circuitry, or in computer software, firmware, or hardware, including the structural means disclosed in this specification and structural equivalents thereof, or in combinations of them. The techniques can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple computers. A computer program (also known as a program, software, software application, or code) can be written in any form of programming language, including compiled or interpreted languages, and it can be deployed in any form, including as a standalone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification can be performed by one or more programmable processors executing one or more computer programs to perform the described functions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, the processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, aspects of the described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The techniques can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

One computer-implemented modeling algorithm is described herein (namely, principal component analysis (PCA)), although such algorithms themselves are generally outside the scope of the present invention. Other software-based modeling algorithms can also be utilized, alone or in combination, such as classification or decision trees, elastic net analysis, linear and polynomial support vector machines (SMV), shrunken centroids, random forest algorithms, support vector machines or neural networks.

### EXAMPLES

The invention is further described in the following examples, which do not limit the scope of the invention described in the claims.

### Methods

The following methods and materials were used in Example 1, below.

### TCC130 & TCGA BLCA

Bladder samples were collected and processed using the Total Cancer Care (TCC) biobanking protocol (Fenstermacher et al., The Cancer Journal 2011;17:528-36. doi:10.1097/PPO.0b013e318238216e) and arrayed on a custom Affymetrix HuRSTA GeneChip (GPL10379, Affymetrix, Santa Clara, CA). The GeneChips CEL file was normalized using interative rank-order normalization (IRON) (Welsh et al., BMC Bioinformatics 2013;14:153. doi:10.1186/1471-2105-14-153) and log2 transformed before further analysis. The RNAseq gene expression for the TCGA bladder tumors (BLCA) was extracted from the normalized and de-batched PanCan RNAseq file available at Genomic Data Commons (GDC, gdc.cancer.gov/aboutdata/publications/pancanatlas) and loge transformed. Survival data was based on the publication by Liu et al. (Cell 2018;173:400-11).

### 12-Chemokine Score

The 12-chemokine (12CK) score was calculated by using the first principal component (PC1) from a principal component analysis (PCA) using the 12-selected genes (CCL2, CCL3, CCL4, CCL5, CCL8, CCL 18, CCL 19, CCL21, CXCL9, CXCL10, CXCL11, and CXCL13). For the TCC130 dataset, the probeset with the highest mean was selected if several probesets was available for one gene. The 12CK score was normalized to unit variance and scores >1 were considered 12CK-High (n=25)

Analysis was performed on deidentified data. The tumor tissue was arrayed on a custom Affymetrix GeneChip. The expression data for the 150 individual patients was normalized using IRON (Welsh et al., BMC Bioinformatics. 2013;14(1):153) and expression data, in log2 units, for the 12 chemokine genes (CCL2, CCL3, CCL4, CCL5, CCL8, CCL18, CCL19, CCL21, CXCL9, CXCL10, CXCL11, and CXCL13, represented in 13 probesets) were extracted, with the following probes:

| Probeset | GeneSymbol |
|---|---|
| merck-NM_002988_at | CCL18 |
| merck-NM_006274_at | CCL19 |
| merck-NM_002982_at | CCL2 |
| merck-NM_002989_at | CCL21 |
| merck-D63785_x_at | CCL3 |
| merck-NM_002984_at | CCL4 |
| merck-NM_002985_at | CCL5 |
| merck-NM_005623_at | CCL8 |
| merck-NM_001565_at | CXCL10 |
| merck-NM_005409_at | CXCL11 |
| merck2-NM_005409_at | CXCL11 |
| merck-NM_006419_at | CXCL13 |
| merck-NM_002416_at | CXCL9 |

A 12-Chemokine GES was identified previously in colorectal cancer from a metagene grouping with overwhelming enrichment for immune-related and inflammation-related genes; see WO 2011/094483 and WO 2015/157623).

A principal component analysis (PCA) was performed from these 13 probe sets. Principal component analysis (PCA) is a technique that reduces a high-dimensional dataset to a low-dimensional dataset while retaining most of the variation in the data (Jolliffe IT. Principal Component Analysis. 2ed. New York: Springer; 2002). The new low-dimensional dataset is created by the PCA-derived principal components also called scores. These are a linear combination of all variables, where the loadings describe the importance of the original variable for each principal component. The first principal component describes most of the variance and is often considered the most important principal component, while the following principal components shows a decreasing amount of explained variance. The results of a PCA models are frequently visualized in score and loading plots. The score plot is related to the samples and shows which samples are similar to each other, groupings between classes of samples and also outliers. The loading plot shows which variables are important for the results seen in the score plot and also which variables are similar to each other. PCA was performed using Evince V2.5.5 (UmBio AB, Umeå, Sweden). Each variable was normalized to unit variance prior to PCA. PC1, representing the most variability (˜58.5%) within the samples, was used to represent the chemokine signature. Based on PC1, samples with values above or below the median were identified as the high and low expressers of the chemokine signature, respectively.

### Deconvolution of Infiltrating Immune Cells

xCell (Aran et al., Genome Biol. 2017 Nov 15;18(1):220), a gene signature-based method was used to enumerate the amount of infiltrating immune cells. Signatures of interest were those associated with CD4+ and CD8+ T lymphocytes, aDC, B lymphocytes, M1 macrophage, NK cells, CD8+ Tem, CD4+ Tem, and memory B cells.

### Pathologic Analysis of Tissue Sections

Slides from 43 cases of cystoprostatectomy specimens for primary urothelial cancer of the bladder were retrieved from the Moffitt Cancer Center Anatomic Pathology Division's repository. The specimens were fixed in 10% formalin and pertinent sections from the tumor and adjoining foci paraffin embedded (FFPE). Histologic slides were prepared from sequential cuts through FFPE bladder tumor specimens. These cuts were adjacent to tissue submitted for mRNA microarray analysis. 12CK scores were generated and normalized to unit variance from mRNA microarray analysis as previously described. Twenty-five (n=25) patients had 12CK scores >1 and were classified as 12CK-High (range, 1.01-2.15), of which 22 (88%) underwent histological evaluation. Twenty-one (n=21/105, 20%) samples corresponding to the samples with the lowest 12CK scores (range, −2.37 to −1.08) also underwent histologic evaluation. Hematoxylin and Eosin (H&E) stained sections from tumor blocks were evaluated for the presence of lymphocytes. To further characterize the TME, immunohistochemical stains were performed on one representative tissue section from each case using the avidin-biotin complex method with retrieval under high pH. Prediluted monoclonal antibodies to CD4, CD8, CD20, and LAMP3 (Ventana Medical Systems, Tucson, AZ, USA) were used. The IHC-stained slides were analyzed under brightfield microscopy and a semiquantitative scoring provided. For no staining in the immune cells score provided was 0, if <25% of immune cells stained score provided was 1, if 25-50% of immune cells stained score provided was 2 and if >50% of the immune cells stained score provided was 3.

Lymphoid cells present around the tumor were classified in to 3 types as described by Kroeger et al. (Clinical Cancer Research 2016;22:3005-15). Type I lymphoid aggregates contained approximately 20-50 cells which were composed of a mixture of CD4 T cells, CD8 T cells, B cells, and occasional dendritic cells. Type II lymphoid aggregates were composed of similar cell make up but were larger in size comprising of 100-1000 cells. Type III lymphoid aggregates represented fully developed tertiary lymphoid structures (TLS) containing prominent germinal centers within lymphoid follicles. The TLS comprised of B cells, discrete T cell zones with CD4, and CD8 cells, and aDC, and high endothelial venule like vessels.

A dedicated genitourinary pathologist performed the pathological review and was blinded to the patients' 12-CK scores. The Fisher's exact test was used to test the associations between the 12-CK scores and the type of lymphoid aggregate by a biostatistician.

### Statistical Analysis

All calculations were performed in MATLAB (R2019b). Kaplan-Meier plots and log rank tests were performed using MatSurv (Creed et al., Joss 2020;5:183). All tests are two-tailed Students t-test assuming unequal variance.

Univariate and Multivariable Cox regression models were used to establish Hazard Rations for overall survival (OS) and progression-free survival (PFS) by a biostatistician (YK) while Disease-specific survival (DSS) used competing-risk subdistribution hazard regression model. Variables included in these models were normalized 12-CK score, age at cystectomy, pathologic T and N stage, neoadjuvant chemotherapy and adjuvant chemotherapy.

### Example 1. Prognostic and Predictive Implications of the 12-Chemokine Score in Muscle Invasive Bladder Cancer

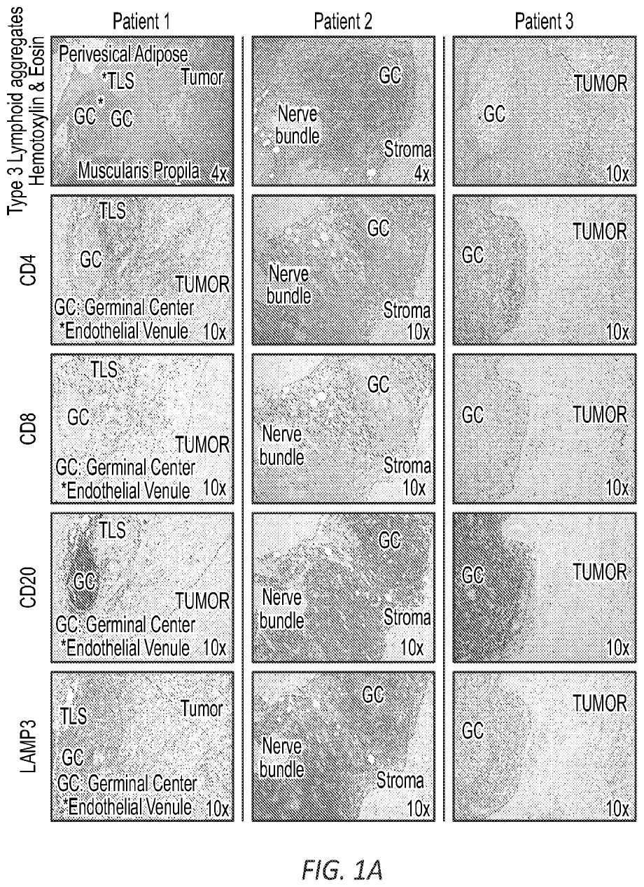

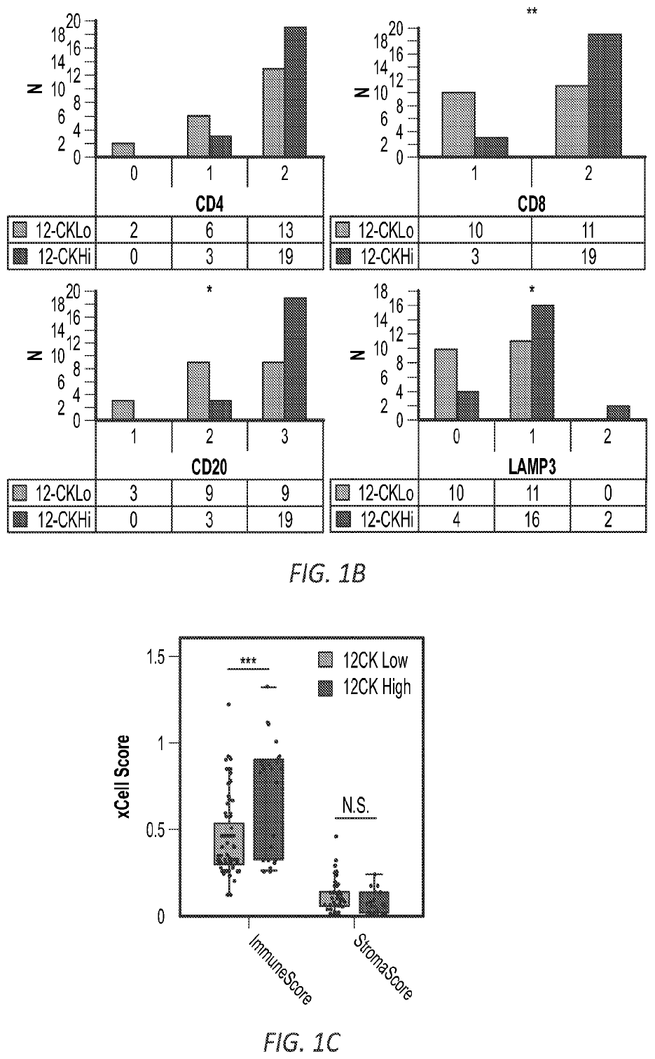

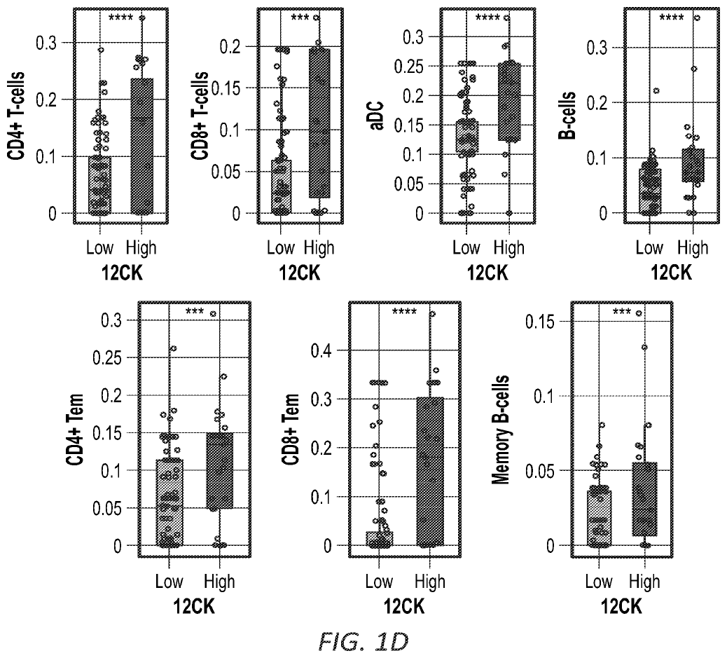

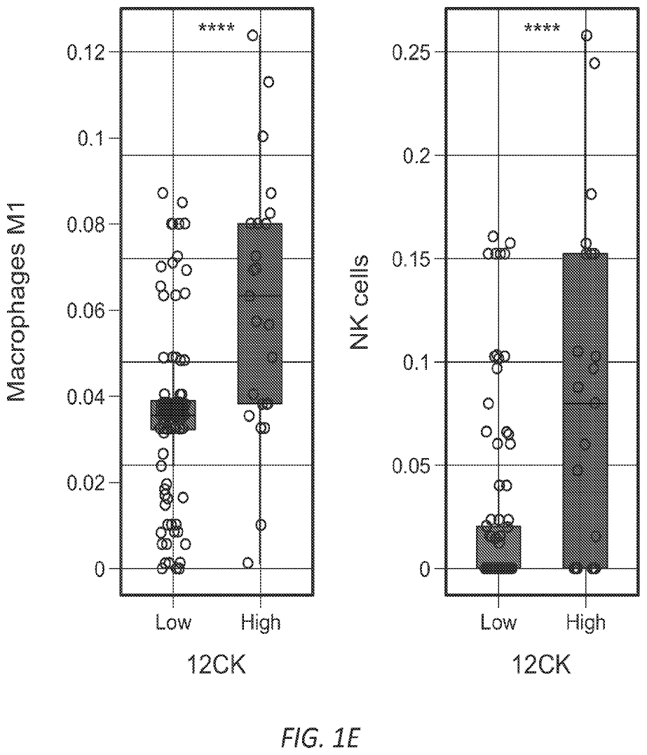

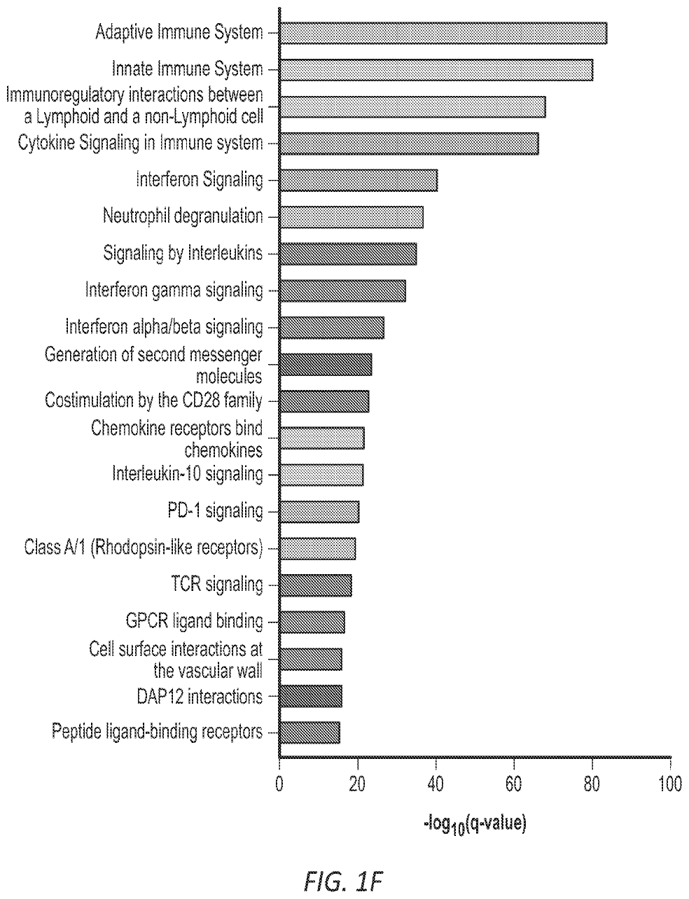

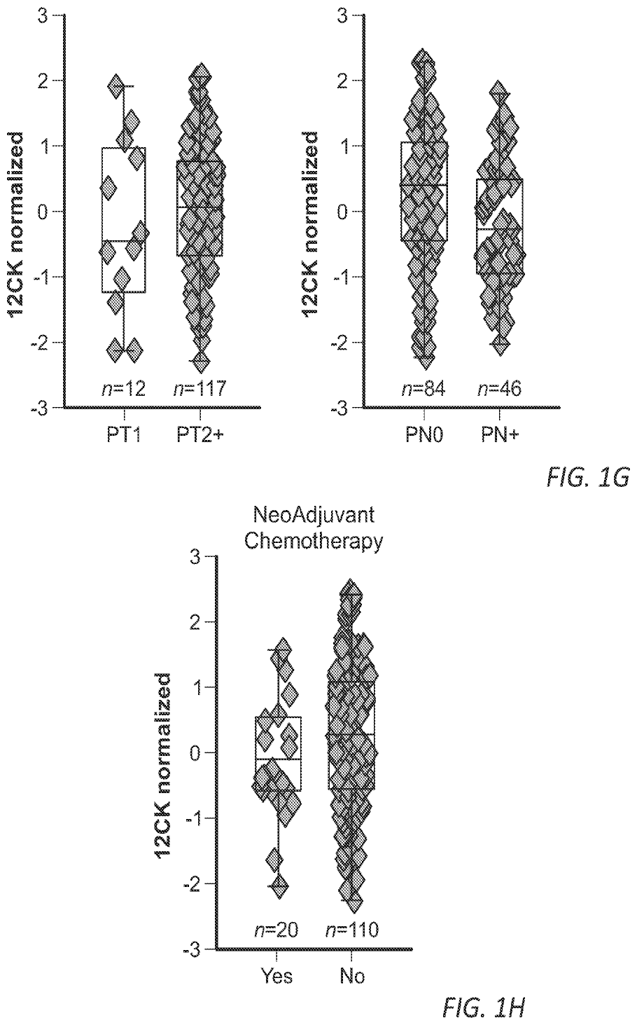

Following IRB approval, 130 MIBC samples were arrayed on Affymetrix HuRSTA GeneChip (GPL10379) microarrays (Affymetrix, Santa Clara, CA) and the 12CK scores were assessed. Baseline clinicopathologic variables were reviewed and summarized (Table 1). 12CK scores were normalized to unit variance, using 12CK>1 to denote 12CK-High (n=24, 18.5%). Immunohistochemistry (IHC) was performed using antibodies to CD4, CD8, CD20, and LAMP3, and cellular densities were quantified using the H-score. Within their TME, 12-CK High tumors consistently exhibited a more robust immuno-environment marked by a higher density of CD4+ T cells (p=0.002), CD8+ T cells (p<0.001), and CD20+ B cells (p=0.002), but not LAMP3+ aDC (p=0.3) (FIG. 1A). Additionally, a more robust immuno-environment was seen in 12CK-High tumors, consisting of increased infiltration of CD4+ T lymphocytes (p=0.1), CD8+ T lymphocytes (p=0.02), activated dendritic cells (aDC) expressing DC-LAMP3 (p=0.047), and B lymphocytes (p=0.006) found on immunohistochemical (IHC) evaluation (FIG. 1B). Next, we systematically compared the presence of TLS and the associated immune cellular infiltration in 12CK-High vs. 12CK-Low tumors and classified them into Type I-III as previously described (10). Type III TLS, consisting of prominent B-cell follicles with germinal center-like structures and discrete T-cell zones10 were found in 7/22 12CK-High vs. 0/21 12CK-Low tumors (Fisher's Exact p=0.009). Of the 23 12-CK High tumor samples evaluated, there were 11 with Type III TLS. In contrast, Type III TLS was only found in 1 of the 21 12-CK Low tumor samples (p<0.002) (50).

TABLE 1
Baseline Clinical and pathologic characteristics
of 130 patient cohort

| Variable | N | % |
|---|---|---|
| Age (median) | 71 | (IQR, 62-77) |
| Normalized 12-CK score | 0.55 | (IQR, −0.69-0.79) |
| Gender | | |
| Male | 105 | 80.8 |
| Female | 25 | 19.5 |
| Chemotherapy | | |
| Neoadjuvant | 20 | 15.4 |
| Adjuvant | 50 | 38.5 |
| pT Stage | | |
| <T2 | 14 | 10.8 |
| T2 | 26 | 20.0 |
| ≥T3 | 90 | 69.2 |
| pN Stage | | |
| X | 3 | 2.3 |
| 0 | 84 | 64.6 |
| 1 | 14 | 10.8 |
| 2 | 29 | 22.3 |
| Vital Status | | |
| Alive with Disease | 1 | 0.8 |
| Dead of Other Causes | 44 | 33.8 |
| Dead of Disease | 57 | 43.8 |
| No evidence of Disease | 28 | 21.5 |

To further explore the immunologic correlates of high 12CK expression, a cell type enrichment analysis from gene expression method (xCell) was used to deconvolute the makeup of the TME in the mRNA microarray data. Cell type enrichment scores across 64 immune and stromal cell types were obtained. Although stromal scores were similar between the two cohorts, immune scores representing the overall immune cell content were markedly higher in the 12-CK High tumors (FIG. 1C). Corroborating the IHC findings, the 12CK-High tumors highly expressed transcriptomic signatures associated with CD4+ T lymphocyte, CD8+T lymphocyte, aDC, and B lymphocytes (FIG. 1D, top). Furthermore, M1 macrophage, NK cells, CD8+ Tem, CD4+ Tem, and memory B cells were enriched in 12CK-High tumors, suggesting both a heightened innate and adaptive immune response (FIG. 1D, bottom and FIG. 1E). On gene set enrichment analysis, REACTOME signatures for immune activation including TCR signalling, CD28 co-stimulation, IFN-γ response, IFN-α response, cytokine signalling, and chemokine receptor binding were found to be associated with 12CK-High tumors (FIG. 1f). The 12-CK score was not found to correlate with traditional prognostic indicators such as pathologic T-staging (FIG. 1G, right) or N-staging (FIG. 1G, right). Moreover, no differences were observed in the 12-CK score amongst tumors that were treatment naïve vs. those collected following neoadjuvant chemotherapy (FIG. 1H).

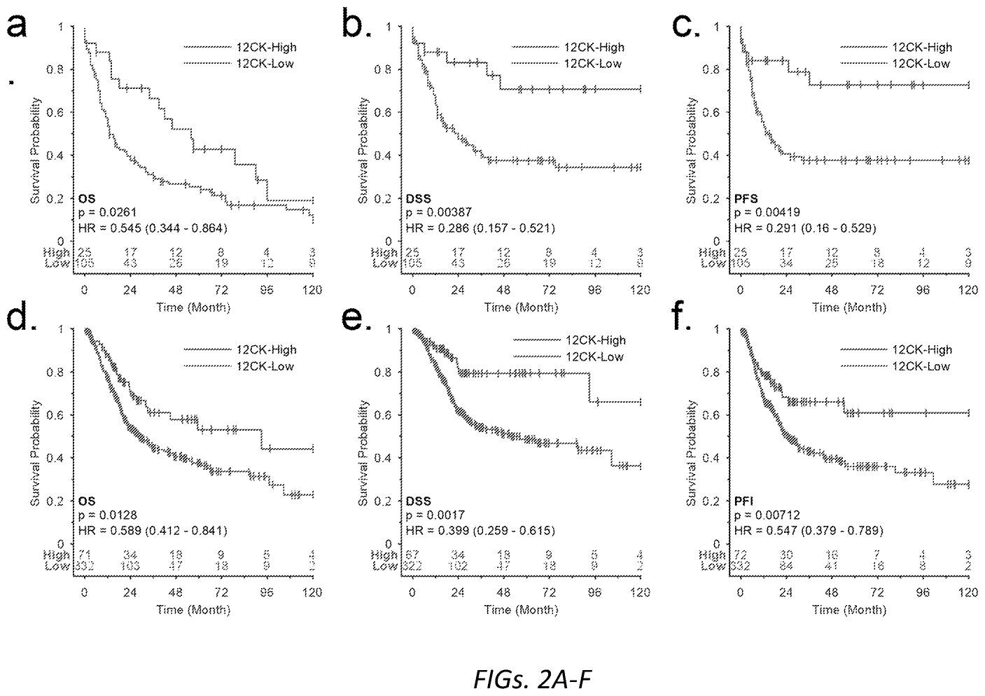

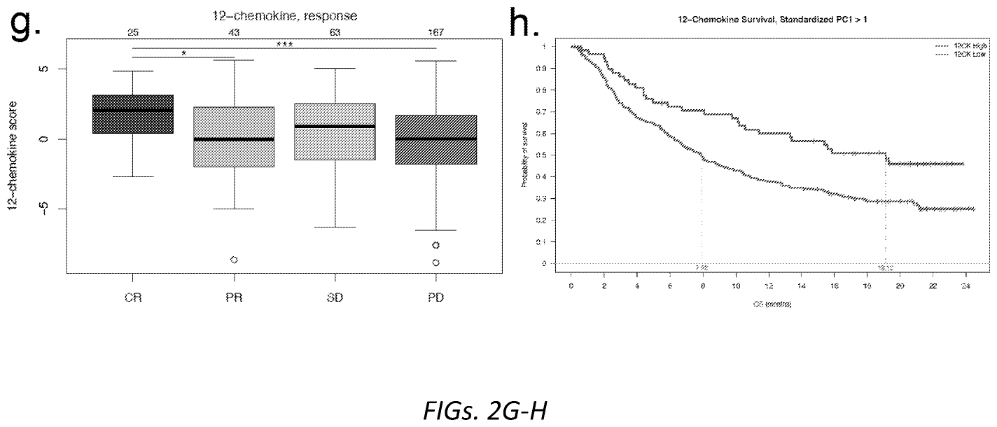

Kaplan-Meier survival analyses of our internal cohort revealed improved progression-free survival (PFS (HR 0.29, p=0.004), disease-specific survival (DSS, HR 0.29, p=0.004), and overall survival (OS, HR 0.55, p=0.03) amongst 12CK-High patients. (FIGS. 2A-C). On multi-variable analysis incorporating age, pathologic T and N stage, and use of neoadjuvant chemotherapy, high 12-CK score was found to independently prognosticate improved PFS (HR 0.77, 95% CI 0.62-0.95, p=0.01), DSS (HR 0.63, 95% CI 0.49-0.81, p=0.0003), and OS (HR 0.81, 95% CI 0.65-0.998, p=0.048). (Tables 2-4).

TABLE 2
Multivariable Cox Proportional Model analysis for
progression-free survival (PFS) identifying that
only 12-CK score was associated with improved OS.

| | Hazard | (95% | P |
|---|---|---|---|
| Variable | Ratio | CI) | Value |
| Normalized 12-CK | 0.77 | (0.61-0.94) | 0.01 |
| Age | 1.01 | (0.99-1.04) | 0.29 |
| Pathologic T Stage | | | |
| <pT2 (ref) | — | — | — |
| pT2 | 1.31 | (0.57-3.03) | 0.52 |
| >pT2 | 1.65 | (0.77-3.51) | 0.38 |
| Pathologic N Stage | | | |
| 0 (ref) | — | — | — |
| 1 | 1.18 | (0.61-2.29) | 0.42 |
| 2 | 1.43 | (0.84-2.46) | 0.19 |
| Neoadjuvant Chemo | | | |
| No (ref) | — | — | — |
| Yes | 0.76 | (0.41-1.41) | 0.38 |
| Adjuvant Chemo | | | |
| No (ref) | — | — | — |
| Yes | 1.21 | (0.75-1.97) | 0.44 |

TABLE 3
Multivariable Cox Proportional Model analysis for
disease specific survival (DSS) identifying that
12-CK score was associated with improved DSS

| | Hazard | (95% | P |
|---|---|---|---|
| Variable | Ratio | CI) | Value |
| Normalized 12-CK | 0.63 | (0.49-0.82) | 0.0005 |
| Age | 0.998 | (0.97-1.03) | 0.88 |
| Pathologic T Stage | | | |
| <pT2 (ref) | — | — | — |
| pT2 | 2.11 | (0.61-7.28) | 0.24 |
| >pT2 | 2.26 | (0.71-7.19) | 0.39 |
| Pathologic N Stage | | | |
| 0 (ref) | — | — | — |
| 1 | 1.46 | (0.72-2.998) | 0.30 |
| 2 | 1.53 | (0.84-2.79) | 0.16 |
| Neoadjuvant Chemo | | | |
| No (ref) | — | — | — |
| Yes | 0.67 | (0.31-1.46) | 0.32 |
| Adjuvant Chemo | | | |
| No (ref) | — | — | — |
| Yes | 1.75 | (0.988-3.11) | 0.055 |

TABLE 4
Multivariable Cox Proportional Model analysis
for overall survival (OS) identifying that only
12-CK score was associated with improved OS

| | Hazard | (95% | P |
|---|---|---|---|
| Variable | Ratio | CI) | Value |
| Normalized 12-CK | 0.79 | (0.63-0.98) | 0.034 |
| Age | 1.02 | (0.99-1.04) | 0.20 |
| Pathologic T Stage | | | |
| <pT2 (ref) | — | — | — |
| pT2 | 1.47 | (0.62-3.52) | 0.39 |
| >pT2 | 1.89 | (0.86-4.16) | 0.12 |
| Pathologic N Stage | | | |
| 0 (ref) | — | — | — |
| 1 | 1.16 | (0.60-2.25) | 0.65 |
| 2 | 1.61 | (0.94-2.76) | 0.08 |
| Neoadjuvant Chemo | | | |
| No (ref) | — | — | — |
| Yes | 0.69 | (0.37-1.28) | 0.24 |
| Adjuvant Chemo | | | |
| No (ref) | — | — | — |
| Yes | 1.16 | (0.72-1.87) | 0.55 |

To externally validate the prognostic value of the 12CK score, we interrogated data from TCGA, and found similar improvements in PFS (HR 0.55, p=0.007), DSS (HR 0.40, p=0.002), and OS (HR 0.59, p=0.01) in 12CK-High patients (FIG. 2d-f). Together, these findings highlight the important favourable prognostic implications of high 12CK-High scores in surgically treated MIBC patients. Furthermore, extrapolating from the association between the presence of TLS and improved outcomes in ICB trials in other cancer types, we examined the association between high 12CK scores and response to atezolizumab in the IMVIGOR 210 study.² In this single-arm, phase 2 trial, patients with inoperable locally advanced or metastatic bladder cancer with disease progression following platinum-based chemotherapy were enrolled and treated with intravenous atezolizumab (1200 mg, given every 3 weeks). In 310 patients receiving atezolizumab treatment, 15% objective response was observed overall, with ongoing responses observed in 84% of the responders. Stratified by treatment response, the complete responders (CR) exhibited significantly higher 12-CK scores than all other groups (FIG. 2G). Strikingly, the 12-CK^{Hi} signature conferred a median overall survival benefit of almost 1 year in the atezolizumab-treated patients (FIG. 2H).

### REFERENCE

1. Necchi A, Raggi D, Gallina A, et al. Updated Results of PURE-01 with Preliminary Activity of Neoadjuvant Pembrolizumab in Patients with Muscle-invasive Bladder Carcinoma with Variant Histologies. Eur Urol. 2020;77(4):439-446.

2. Rosenberg J E, Hoffman-Censits J, Powles T, et al. Atezolizumab in patients with locally advanced and metastatic urothelial carcinoma who have progressed following treatment with platinum-based chemotherapy: a single-arm, multicentre, phase 2 trial. Lancet (London, England). 2016;387(10031):1909-1920.

3. Mariathasan S, Turley S J, Nickles D, et al. TGFβ attenuates tumour response to PD-L1 blockade by contributing to exclusion of T cells. Nature. 2018;554(7693):544-548.

4. Cabrita R, Lauss M, Sanna A, et al. Tertiary lymphoid structures improve immunotherapy and survival in melanoma. Nature. 2020;577(7791):561-565.

5. Helmink B A, Reddy S M, Gao J, et al. B cells and tertiary lymphoid structures promote immunotherapy response. Nature. 2020;577(7791):549-555.

6. Petitprez F, de Reyniès A, Keung E Z, et al. B cells are associated with survival and immunotherapy response in sarcoma. Nature. 2020;577(7791):556-560.

7. Sautes-Fridman C, Petitprez F, Calderaro J, Fridman W H. Tertiary lymphoid structures in the era of cancer immunotherapy. Nat Rev Cancer. 2019;19(6):307-325.

8. Coppola D, Nebozhyn M, Khalil F, et al. Unique ectopic lymph node-like structures present in human primary colorectal carcinoma are identified by immune gene array profiling. Am J Pathol. 2011;179(1):37-45.

9. Messina J L, Fenstermacher D A, Eschrich S, et al. 12-Chemokine gene signature identifies lymph node-like structures in melanoma: potential for patient selection for immunotherapy? Scientific reports. 2012;2:765.

10. Kroeger D R, Milne K, Nelson B H. Tumor-Infiltrating Plasma Cells Are Associated with Tertiary Lymphoid Structures, Cytolytic T-Cell Responses, and Superior Prognosis in Ovarian Cancer. Clin Cancer Res. 2016;22(12):3005-3015.

11. Sharma P, Shen Y, Wen S, et al. CD8 tumor-infiltrating lymphocytes are predictive of survival in muscle-invasive urothelial carcinoma. Proc Natl Acad Sci U S A. 2007;104(10):3967-3972.

12. Necchi A, Raggi D, Gallina A, et al. Impact of Molecular Subtyping and Immune Infiltration on Pathological Response and Outcome Following Neoadjuvant Pembrolizumab in Muscle-invasive Bladder Cancer. European Urology. 2020.

13. van Dijk N, Funt S A, Blank C U, Powles T, Rosenberg J E, van der Heijden M S. The Cancer Immunogram as a Framework for Personalized Immunotherapy in Urothelial Cancer. Eur Urol. 2019;75(3):435-444.

14. Wang L, Saci A, Szabo P M, et al. EMT—and stroma-related gene expression and resistance to PD-1 blockade in urothelial cancer. Nat Commun. 2018;9(1):3503.

15. FDA U. FDA/Merck, Sharpe & Dohme COMBINED Briefing Information for the Dec. 17, 2019 Meeting of the Oncologic Drugs Advisory Committee (PM Session). In: FDA U, ed: fda.gov; 2019.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has muscle-invasive bladder cancer (MIBC), the method comprising:
obtaining cells from the tumor;
determining expression levels of CCL2, CCL3, CCL4, CCL5, CCL8, CCL18, CCL19, CCL21, CXCL9, CXCL10, CXCL11, and CXCL13 in the tumor cells;
comparing the tumor gene expression levels to reference gene expression levels;
selecting for the subject a first line treatment comprising surgical resection and immunotherapy and/or adjuvant chemotherapy if tumor gene expression levels are above the reference gene expression levels.

2. The method of claim 1, wherein determining gene expression levels comprises determining protein levels.

3. The method of claim 1, wherein determining gene expression levels comprises determining mRNA levels.

4. The method of claim 1, wherein the immunotherapy comprises administering to the subject dendritic cells or peptides with adjuvant, a DNA-based vaccine, cytokines, cyclophosphamide, anti-interleukin-2R immunotoxin, or a checkpoint inhibitor.

5. The method of claim 4, wherein the checkpoint inhibitor is an inhibitor of PD-1 signaling, preferably an antibody that binds to PD-1, CD40, or PD-L1, or an inhibitor of Tim3 or Lag3, preferably an antibody that binds to Tim3 or Lag3, or an antibody that binds to CTLA-4.

6. The method of claim 1, wherein the adjuvant chemotherapy comprises administering to the subject a platinum-based chemotherapeutic, e.g., cisplatin, carboplatin or oxaliplatin; paclitaxel, and/or gemcitabine; cisplatin/carboplatin (GC); Pembrolizumab; or methotrexate, vinblastine, doxorubicin, and cisplatin (MVAC).

7. The method of claim 1, wherein the subject is a human.

8. The method of claim 1, wherein comparing expression levels comprises calculating an expression score that is a weighted average of the mRNA expression levels of CCL2, CCL3, CCL4, CCL5, CCL8, CCL18, CCL19, CCL21, CXCL9, CXCL10, CXCL11, and CXCL13, and comparing the expression score to a reference expression score.
